(19) United States
(12) Patent Application Publication
(10) Pub. No.: US 2023/0119454 A1
(43) Pub. Date: Apr. 20, 2023

(54) SYSTEM AND METHOD FOR ASSESSING CONDITIONS OF VENTILATED PATIENTS

(71) Applicant: Vyaire Medical, Inc., Mettawa, IL (US)

(72) Inventors: Christopher M. VARGA, Laguna Hills, CA (US); Hasnain SOMJI, Mettawa, IL (US); Alejando Jose VILLASMIL, Mettawa, IL (US); RICH KUSLEIKA, Mettawa, IL (US)

(21) Appl. No.: 17/914,312

(22) PCT Filed: Mar. 23, 2021
(86) PCT No.: PCT/US2021/023765

§ 371 Date: Sep. 23, 2022

Related U.S. Application Data
(60) Provisional application No. 62/994,253, filed on Mar. 24, 2020.

(51) Int. Cl. A61M 16/00; G16H 50/70; G16H 40/63

(52) U.S. Cl. CPC A61M 16/024; G16H 50/70; G16H 40/63; A61M 2205/3306; A61M 2205/3303; A61M 2230/63; A61M 2230/60; A61M 2205/3375

(57) ABSTRACT

The disclosed system receives various physiological as well as physical information concerning a patient, and operational data from a ventilation device and medication delivery device, and provides the physiological and physical information, together with the operational data, to a neural network configured to analyze the information and data. The system receives, from the neural network, an assessment classification of the patient corresponding to at least one of a pain assessment, a sepsis assessment, and a delirium assessment of the patient based on providing to the neural network the determined physiological state of the patient, the determined physical state of the patient, the determined operational mode of the ventilator, the medication delivery information, and the received diagnostic information for the patient, and adjusts, based on the assessment classification, a ventilation parameter that influences the operational mode of a ventilator providing ventilation to the patient.

### BACKGROUND FIELD

The subject technology addresses deficiencies commonly encountered in hospital care with regard to assessing conditions of ventilated patients and adjusting ventilation parameters to stabilize such patients.

### SUMMARY

The subject technology addresses deficiencies commonly encountered in hospital care and medical care involving assessment of mechanically ventilated patient status with respect to pain levels, sepsis, delirium, intensive-care-unit (ICU) acquired weakness, post-intensive care syndrome, and appropriate choice of medications and their dosing. Aspects of the subject technology specifically address issues encountered by caregivers when they attempt to combine objective and subjective patient data to provide assessment or status of mechanically ventilated patients with respect to the abovementioned conditions/issues. For example, present state-of-the-art methods for assessing patient pain level involves combining objective data which is available from the ventilator (e.g. patient-ventilator asynchrony or presence of alarms due to coughing) with subjective data regarding comfort or relaxation. To assess comfort, a caregiver will independently decide by looking at a patient's face whether they look tense or whether they are grimacing. For muscle tension, they will perform a passive movement of a patient limb and make a personal determination of how much tension or resistance is encountered during the movement. By scoring each activity subjectively, the caregiver estimates the level of pain the patient is experiencing, and may assign that level to a subjective score. The effectiveness of such a strategy for assessment is highly dependent upon both the skill level or experience of the caregiver in view of any physiological data available to the caregiver at the time of assessment.

To assess or monitor for sepsis in a ventilated patient, a caregiver performs an assessment which combines data available from a ventilator providing ventilation to the patient, and adjacent monitors that provide, for example, work-of-breathing, patient core temperature and blood pressure. The assessment may also take into consideration subjective data related to how a patient looks or whether they are experiencing rigors (exaggerated shivering). These subjective measures are assessed visually by the caregiver and are subject to the same limitations described above for pain levels. For assessment of delirium, a caregiver may combine objective data related to drug dosing alongside subjective measures which involve a conversation between the caregiver and the patient (e.g. questions and answers). Delirium can also be subjectively characterized by observing erratic body movements and visible patient agitation. For assessment of ICU-acquired weakness, a caregiver may make an assessment involving objective measures such as ventilator settings, duration of ventilation, respiratory effort (e.g. spontaneous breathing rate) alongside subjective measures such as manual muscle strength testing. Assessment or prediction of post-intensive care unit syndrome (PICS) involves assessment of ventilation, delirium, pain, sepsis, and ICU-acquired weakness together, yet there is currently no comprehensive mechanism or system to provide this data in an objective way to a caregiver to enable them to prepare a patient for the care they will need after leaving the ICU.

Additionally, selection of the most appropriate medications for a ventilated patient (e.g. sedatives, analgesics, hypnotics, antibiotics) presently falls upon the practitioner; that is, the practitioner makes a clinical assessment of the individual ventilated patient and then selects medications which are appropriate based on numerous factors including the patient's condition as well as their own experience or clinical judgment. Selection of the best medications for an individual based upon a large number of contributing and competing factors can be equally as difficult as assessing patient conditions as described above.

Accordingly, there is a need for a system which can provide integrated, objective measures of the inputs discussed above which are required to make repeatable and accurate assessments of patient conditions as well as predictions or likelihoods of outcomes such as PICS. Furthermore, there is a need for a system which continually analyzes these assessments alongside available patient data to recommend medications and dosing levels for the ventilated patient. There is also a need for a system which in an automated fashion uses the aforementioned assessments to act on a patient device or devices, setting parameters in a closed-loop fashion in order to better manage pain, delirium, sepsis, ICUAW, and PICS. The subject technology addresses these deficiencies encountered in current care of the mechanically ventilated patient.

According to various implementations, the disclosed system includes one or more processors and a memory. The memory includes instructions stored thereon that, when executed by the one or more processors, cause the one or more processors to perform operations for performing a method of assessing a condition of a ventilated patient and adjusting an operation mode of the ventilator. The method includes receiving diagnostic information for a patient; determining, based on signals received from one or more sensors, a physiological state of the patient; determining an operational mode of a ventilator providing ventilation to the patient; receiving medication delivery information from medication delivery device; activating an imaging device and obtaining image data pertaining to the patient from the imaging device; determining, based on the image data, a physical state of the patient; providing the determined physiological state of the patient, the determined physical state of the patient, the determined operational mode of the ventilator, the medication delivery information, and the received diagnostic information for the patient to a neural network; receiving, from the neural network, an assessment classification of the patient corresponding to at least one of a pain assessment, a sepsis assessment, and a delirium assessment of the patient based on providing to the neural network the determined physiological state of the patient, the determined physical state of the patient, the determined operational mode of the ventilator, the medication delivery information, and the received diagnostic information for the patient; and adjusting, based on the assessment classification, a parameter of the ventilator, wherein adjusting the parameter influences the operational mode of the ventilator. Other aspects include corresponding systems, apparatuses, and computer program products for implementation of the computer-implemented method.

Further aspects of the subject technology, features, and advantages, as well as the structure and operation of various aspects of the subject technology are described in detail below with reference to accompanying drawings.

### DESCRIPTION OF THE FIGURES

Various objects, features, and advantages of the present disclosure can be more fully appreciated with reference to the following detailed description when considered in connection with the following drawings, in which like reference numerals identify like elements. The following drawings are for the purpose of illustration only and are not intended to be limiting of this disclosure, the scope of which is set forth in the claims that follow.

FIGS. 1A and 1B depict example implementations of a pain assessment system, according to various aspects of the subject technology.

FIG. 2 depicts an example implementation of a sepsis assessment system, according to various aspects of the subject technology.

FIG. 3 depicts an example implementation of a delirium assessment system, according to various aspects of the subject technology.

FIG. 4 depicts an example implementation of a ICU-acquired weakness (ICUAW) assessment system, according to various aspects of the subject technology.

FIG. 5 depicts an example implementation of a post-intensive care unit syndrome (PICS) assessment system, according to various aspects of the subject technology.

FIG. 6 depicts an example implementation of a ventilation medication choice and dosing system, according to various aspects of the subject technology.

FIG. 7A depicts an example implementation of an automatic management system, according to various aspects of the subject technology.

FIG. 7B depicts an example implementation of a reinforcement learning algorithm for use by the automatic risk factor management system, according to various aspects of the subject technology.

FIG. 8 is a block diagram illustrating an example system for assessing conditions of ventilated patients and adjusting an operation mode of a ventilator, including a ventilation device, one or more management devices, according to certain aspects of the subject technology.

FIG. 9 depicts an example flow chart of a method of assessing a condition of a ventilated patient and adjusting an operation mode of the ventilator, according to aspects of the subject technology.

FIG. 10 is a conceptual diagram illustrating an example electronic system for assessing conditions of ventilated patients and adjusting an operation mode of a ventilator, according to aspects of the subject technology.

### DESCRIPTION

While aspects of the subject technology are described herein with reference to illustrative examples for particular applications, it should be understood that the subject technology is not limited to those particular applications. Those skilled in the art with access to the teachings provided herein will recognize additional modifications, applications, and aspects within the scope thereof and additional fields in which the subject technology would be of significant utility.

The subject technology comprises a computer-enabled ventilation patient assessment system which integrates and weighs inputs obtained in real time from a mechanical ventilator, alongside additional inputs obtained from integrated measurement devices and components. Objective patient physiological attributes and related measurements are obtained in real time to produce scores, probabilities or likelihoods of a patient state such as pain, sepsis, delirium, ICU-acquired weakness, or PICS. In some implementations, the the assessment system of the subject technology may use these inputs to provide medication recommendations or options.

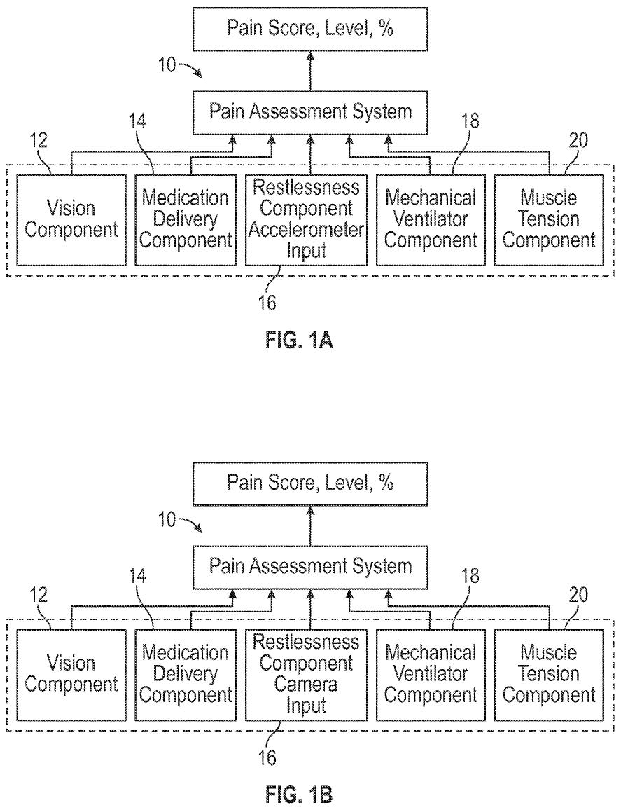

FIGS. 1A and 1B depict example implementations of a pain assessment system, according to various aspects of the subject technology. In the depicted examples, a pain assessment system 10 includes one or more of a mechanical ventilator component 18, a vision component 12, a medication delivery component 14, and a muscle tension measurement component 20, and a body movement (restlessness) component 16. Each component may be implemented by a electromechanical or computer controlled device. For example, the vision component 12 may comprise a camera (not shown) with facial recognition algorithms which monitor a patient and determine a state of a patient's facial expression ranging from relaxed to tense to grimacing.

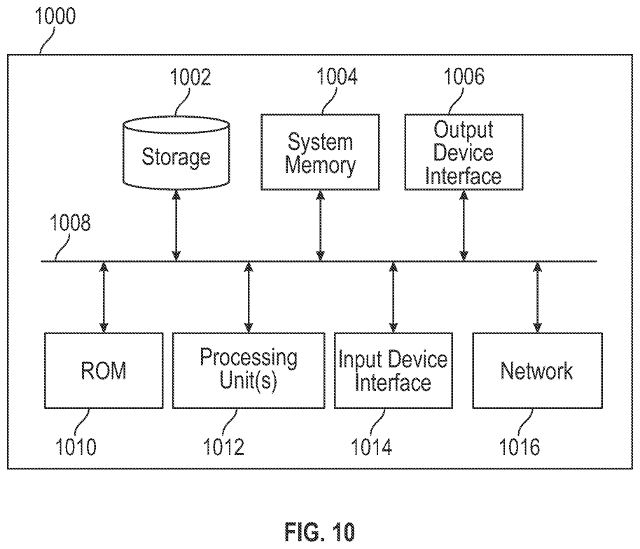

The camera may be positioned in a patient room, or otherwise adjacent to a patient, and configured to capture the face or one or more body portions of the patient. Image data is collected by the camera and received by a central processing unit of the vision component 12 (see, e.g., FIG. 10). The image of the patient, the patient's face, or the body part(s) may be digitally transmitted to a Convolutional Neural Network (CNN) as an input. The CNN may be configured to output the current facial pain state with maps to specific features of the image (face) which contributed to the provided classification.

The medication delivery component 14 may include an infusion pump, or server or other computing device which receives real time information regarding medications administered to the patient. The information may include parameters for a currently administered analgesic or other medication, including but not limited to drug and concentration, dosage, infusion pump settings currently being utilized to administer medications, and medication (e.g. sedation) sedation levels currently being administered. The muscle tension component 20 may include one or more sensors applied to the patient's skin to measure a quantitative level of muscle tension. In some implementations, one or more sensors may include small electrodes placed on the patient's skin in order to record electromyogram (EMG) signals that are, thereafter, fed into a learning algorithm that computes an output consisting of a classification of the patient's muscle tension levels.

A restlessness component 16 may similarly include one or more sensors affixed to the patient's body that provide a sensor output signal as an input to a trained learning algorithm to ultimately output a classification of the patient's restlessness. An example sensor may include a series of accelerometer chips/stickers placed on the patient's arms or legs. Additionally or in the alternative, the restlessness component 16 may include a camera, by which one or more images or video of a portion of the patient may be acquired over a period of time. A number of frames of the images or video may be processed by an image recognition system and provided to a trained Convolutional Neural Network, to output a restlessness score of the patient.

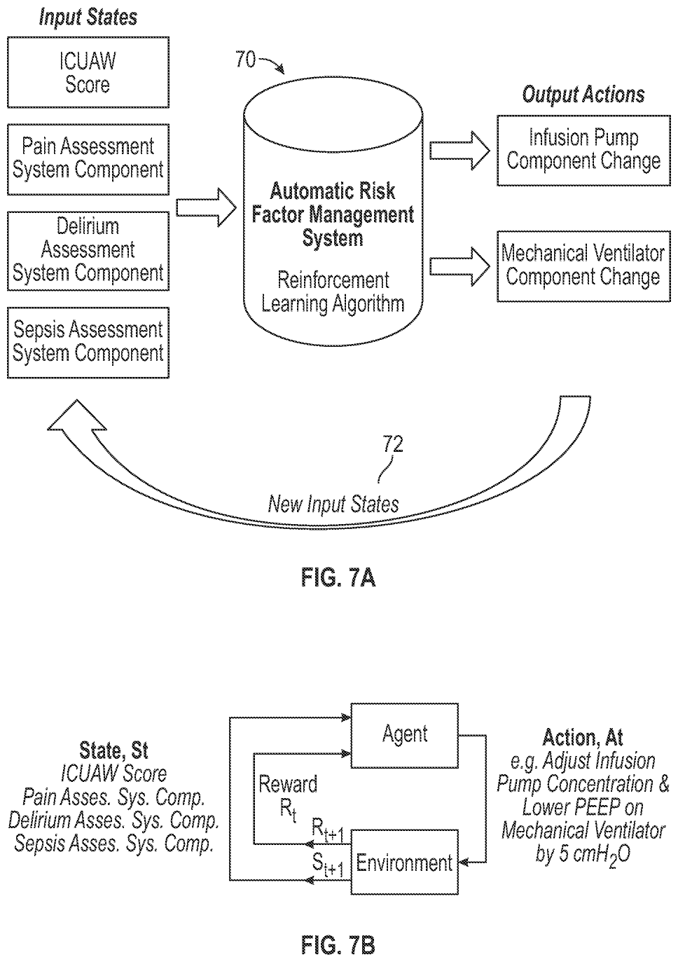

The pain assessment system 10 scores data from each component, utilizing the outputs of each component as inputs, and outputs a pain score, level, or percentage. In some implementations, the pain assessment system 10 includes a trained learning algorithm (e.g. Deep Neural Network) that leverages facial pain classification, medication delivery data, restlessness levels, muscle tension levels, and ventilation parameters as input features, to output a single pain score percentage. The pain assessment system 10 may be configured to receive alarm states and patient-ventilator asynchrony from the mechanical ventilator component 18 to measure patient-ventilation compliance. Upon generating a pain score, level, or percentage, the pain assessment system may deliver this information as a notification to a caregiver, through either a screen on the mechanical ventilator, via a mobile application on the caregiver's device (e.g., device 170), via a web application to a network connected device, or other means of notification. In addition to relaying this output score to an end-user, in some implementations, the generated pain score may be used as an integral input fed back into patient devices (e.g. ventilator, infusion pump, etc.) to adjust key clinical parameters in an automated fashion, as described further with regard to the example implementation(s) of FIG. 7. When used as input to adjust a patient care device, the assessment system may confirm the adjustment and deliver confirmatory messages or check-in notifications to a caregiver through mobile, web, machine or audible means.

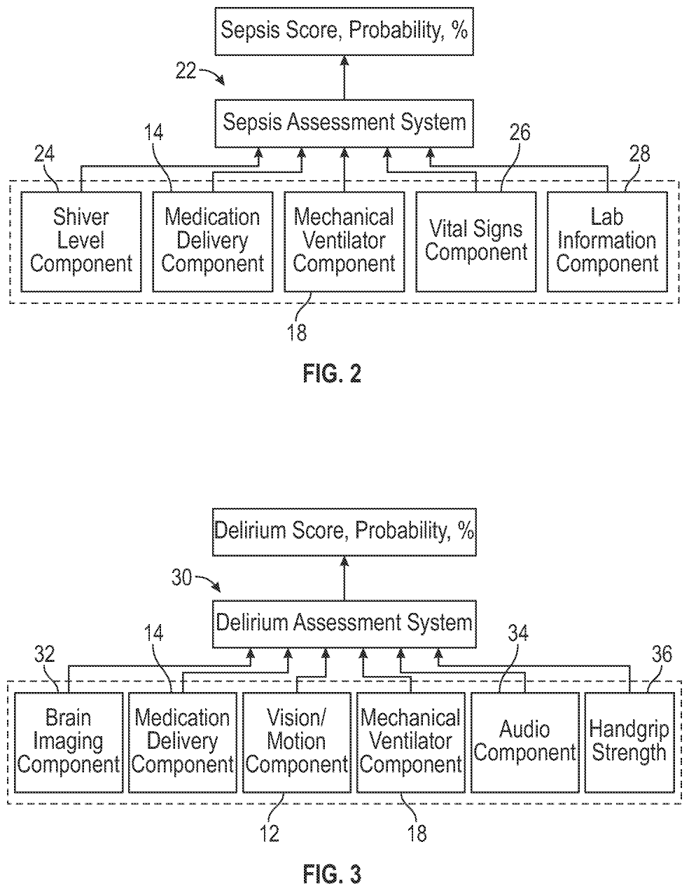

FIG. 2 depicts an example implementation of a sepsis assessment system, according to various aspects of the subject technology. In the depicted example, a sepsis assessment system 22 includes one or more of a shiver level component 24, a medication delivery component 14, mechanical ventilator component 18, a vision component 12, and a vital signs measurement component 26, and a lab information component 28. Each component may be implemented by a electromechanical or computer controlled device. For example, the vision shiver-state component may include an image capture device (e.g., a camera) and/or a series of accelerometers. One or more image frames, together with accelerometer data, may be provided as input to a body recognition algorithm (e.g., a high-frequency motion detection algorithm) configured to determine a shivering state of the patient. The shivering state of the patient may include a value within a range representative of a still or calm to exaggerated shivering (e.g. a numerical score 1-3).

As described previously, the medication delivery component 14 may include an infusion pump, or server or other computing device which receives real time information regarding medications administered to the patient. The information may include parameters for a currently administered analgesic or other medication, including but not limited to drug and concentration, dosage, infusion pump settings currently being utilized to administer medications, and medication (e.g. sedation) sedation levels currently being administered.

The vital signs measurement component 26 may include a monitor or sensor which measures one or more of blood pressure, patient core temperature, heart rate, electrocardiogram (ECG), pulse, or blood oxygen saturation level (e.g., a pulse oximetry sensor). When a time-series waveform measurement is obtained, such as with ECG, the signal may first be characterized based on its features into different buckets, such as, for example, an irregular (arrhythmia) or regular label. Each bucket classification may be associated with a predetermined value which is then provided as input, together with the signals and/or data from the other components, to a classification algorithm of the sepsis assessment system to obtain a final score. The depicted lab information component 28 may comprise a device which obtains or receives blood testing measurements or other patient assays from a hospital information system storing such data. The mechanical ventilator component 18 (e.g., a ventilator or device configured to receive ventilator data) may provide an operational mode of a ventilator providing ventilation to the patient, and/or real-time work-of-breathing (WOB) data to the classification algorithm of the sepsis assessment system to, when processed with the other data, obtain the final sepsis score. The term “operational mode”, as used herein encompasses its plain and ordinary meaning, and includes but is not limited to both the ventilatory mode of operation as well as the specifics of the mode including breath delivery, breath profile, exhalation characteristics, timing, synchrony, and any additional settings relevant to said mode (including, e.g., the quantitative characteristics of the actual mechanical breath delivery and operation).

The sepsis assessment system 22 is configured to score data obtained from each component, and then the scored data is provided as inputs to a final classification algorithm which outputs a sepsis score or probability. In some implementations, this final algorithm is comprised of a simple trained logistic regression algorithm which is capable of outputting sepsis probability. Upon generating a sepsis score or probability, the sepsis assessment system 22 may deliver this information as a notification to a caregiver, through either a screen on the mechanical ventilator, via a mobile application on the caregiver's device (e.g., device 170), via a web application to a network connected device, or other means of notification. In addition to relaying this information to an end-user, in some implementations, the generated sepsis score may be used as an integral input fed back into patient devices (e.g. ventilator, infusion pump, etc.) to adjust key clinical parameters in an automated fashion, as described further with regard to the example implementation(s) of FIG. 7. When used as input to adjust a patient care device, the assessment system may confirm the adjustment and deliver confirmatory messages or check-in notifications to a caregiver through mobile, web, machine or audible means.

FIG. 3 depicts an example implementation of a delirium assessment system, according to various aspects of the subject technology. In the depicted example, a delirium assessment system 30 includes one or more of a mechanical ventilator component 18, a medication delivery component 14, a brain imaging component 32, a vision/motion component 12, an audio component 34, and a handgrip strength component 36. Each component may be implemented by a electromechanical or computer controlled device. For example, the vision/motion component 12 may include a camera and/or a series of accelerometers. In some implementations, the vision component and motion component are separate component devices. One or more image frames (e.g., photo or video), together with accelerometer data, may be provided as input into a behavior recognition algorithm utilizing a trained Convolutional Neural Network configured to determine behaviors or movements from calm to erratic and agitated. In some implementations, the accelerometer data is provided to a high-frequency motion detection algorithm to gauge a patient's erratic movements, for example, by assigning a classification score to the movements. In some embodiments, the vision/motion component captures both large-scale erratic body movements with an accelerometer and small movements of the face and eyes using camera-based vision which can detect fine movements not picked up by accelerometers.

The audio component 34 may include one or more microphones and/or speakers. In some implementations, the audio component 34 may be integrated into the ventilator. The audio component enables the system to ask the patient questions (e.g., via the speaker) and to record answers to determine mental states of the patient ranging from attentive to inattentive or from conscious to unconscious or from organized to disorganized thinking. In some implementations, the patient is monitored by the microphone continuously or periodically over a period of time. Audio data is provided to a algorithm or Natural Language Processing Recurrent Neural Network which leverages the patient's audio response and generates a classification label for each of the categories listed above (i.e. attentiveness, consciousness, organization of thinking). This classification may occur in real time, without user involvement. The brain imaging component 32 may include one or more devices that obtain or receive a series of CT and/or fMRI scans, and analyze the scans using a trained Convolutional Neural Network to detect areas that show signs of ventricular enlargement, brain parenchymal, or chemical/blood flow imbalances to detect pathological changes in brain structure.

The handgrip strength component 36 may comprise a handgrip dynamometer device configured to assess patient response to commands or requests to squeeze the component when the patient is incapable of speaking. Values indicative of the patient's grip may be processed and classified into a predetermined range of values. The device may include dynamometer which obtains a digital output pressure signal which is used to classify the strength of the patient according to a discrete scale. As described previously, the medication delivery component 14 may include an infusion pump, or server or other computing device which receives real time information regarding medications administered to the patient. The information may include parameters for a currently administered analgesic or other medication, including but not limited to drug and concentration, dosage, infusion pump settings currently being utilized to administer medications, and medication (e.g. sedation) sedation levels currently being administered. The delirium assessment system is also configured to utilize oxygenation data as well as alarm states from the mechanical ventilator component to assess patient behaviors and compliance with the ventilator.

The delirium assessment system 30 is configured to score data obtained from each component, and then the scored data is provided as inputs to a final classification algorithm which outputs a delirium score or probability. In some implementations, this algorithm is configured as a trained logistic regression algorithm which is capable of outputting delirium probability. The delirium assessment system scores data from each component, processes the scores collectively, and outputs a delirium score, level or probability. Upon generating a delirium score, level or probability, the delirium assessment system 30 may deliver this information as a notification to a caregiver, through either a screen on the mechanical ventilator, via a mobile application on the caregiver's device (e.g., device 170), via a web application to a network connected device, or other means of notification. In addition to relaying this information to an end-user, in some implementations, the generated delirium score may be used as an integral input fed back into patient devices (e.g. ventilator, infusion pump, etc.) to adjust key clinical parameters in an automated fashion, as described further with regard to the example implementation(s) of FIG. 7. When used as input to adjust a patient care device, the assessment system may confirm the adjustment and deliver confirmatory messages or check-in notifications to a caregiver through mobile, web, machine or audible means.

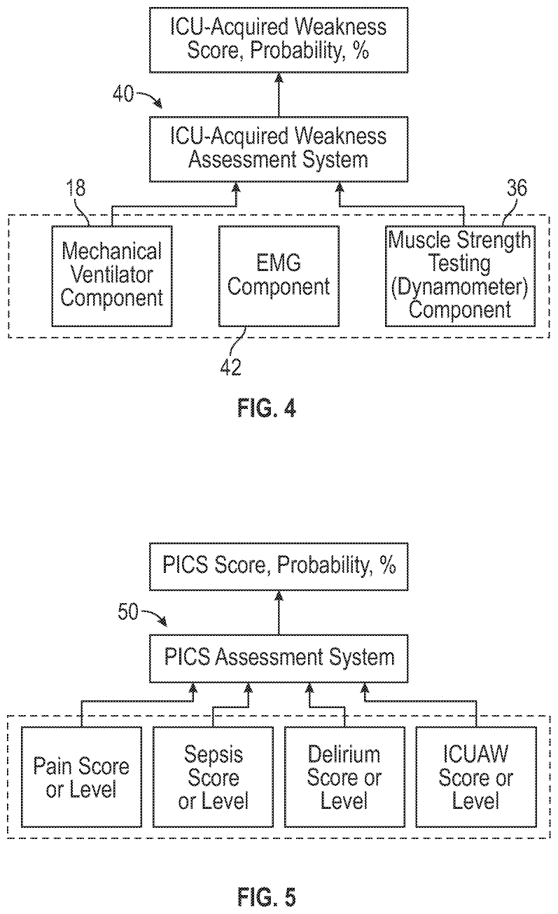

FIG. 4 depicts an example implementation of a ICU-acquired weakness (ICUAW) assessment system 40, according to various aspects of the subject technology. In the depicted example, a ICU-acquired weakness assessment system includes one or more of a mechanical ventilator component, and electromyogram (EMG) component 42, and a muscle strength testing component. Each component may be implemented by a electromechanical or computer controlled device.

As described previously, the handgrip strength component 36 may comprise a handgrip dynamometer device configured to assess patient response to commands or requests to squeeze the component when the patient is incapable of speaking. Values indicative of the patient's grip may be processed and classified into a predetermined range of values. The device may include dynamometer which obtains a digital output pressure signal which is used to classify the strength of the patient according to a discrete scale. The electromyogram (EMG) component 42 may comprise multiple electrodes configured to record muscle activity. Time-series data obtained from the electrodes may be provided to a detection algorithm configured to detect gradual decreases in time-averaged patient muscle tension. The ICU-acquired weakness assessment system 40 utilizes the mechanical ventilator component to monitor and/or measure, or otherwise obtain ventilator settings, duration of ventilation, respiratory effort (e.g. spontaneous breathing rate) or other mechanical ventilation parameters which are markers for patient respiratory effort or strength.

The ICU-Acquired Weakness assessment system 40 scores data from each component and then feeds component outputs as inputs to a ICUAW classification algorithm which outputs an ICU-Acquired Weakness score or probability. In some embodiments, this ICUAW algorithm is a trained logistic regression algorithm configured to output an ICU-Acquired Weakness probability given a series of component inputs. The ICU-acquired weakness assessment system 40 scores data from each component and outputs an ICU-acquired weakness score, level or probability. Upon generating an ICU-acquired weakness score, level or probability, the ICU-acquired weakness assessment system 40 may deliver this information as a notification to a caregiver, through either a screen on the mechanical ventilator, via a mobile application on the caregiver's device, via a web application to a network connected device (e.g., device 170), or other means of notification. In addition to relaying this information to an end-user, in some implementations, the generated ICUAW score may be used as an integral input fed back into patient devices (e.g. ventilator, infusion pump, etc.) to adjust key clinical parameters in an automated fashion, as described further with regard to the example implementation(s) of FIG. 7. When used as input to adjust a patient care device, the assessment system may confirm the adjustment and deliver confirmatory messages or check-in notifications to a caregiver through mobile, web, machine or audible means.

FIG. 5 depicts an example implementation of a post-intensive care unit syndrome (PICS) assessment system, according to various aspects of the subject technology. In the depicted example, a PICS assessment system 50 receives, from the systems described with regard to FIGS. 1 through 4, one or more of a pain score or level, a delirium score or level, a sepsis score or level, and an ICU-acquired weakness score or level. The PICS assessment system 50 applies learned weightings (learned via a linear regression model or through an advanced neural network) to each of these scores to produce a PICS super score or level which indicates the likelihood of a patient to experience PICS. Upon generating a PICS super score, level or probability, the PICS assessment system 50 may deliver this information as a notification to a caregiver through either a screen on the mechanical ventilator, a mobile application, a web application, or other means of notification, in the same manner described previously.

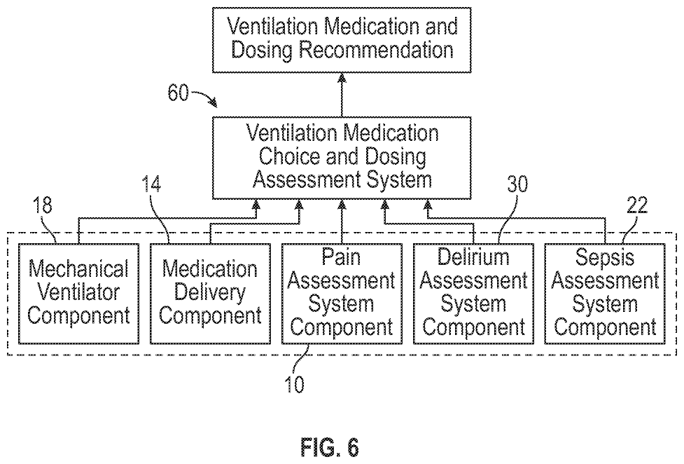

FIG. 6 depicts an example implementation of a ventilation medication choice and dosing system 60, according to various aspects of the subject technology. In the depicted example, a ventilation medication choice and dosing system includes one or more of a mechanical ventilator component 18, a medication delivery component 14 (e.g., an infusion pump), a pain assessment system component 10, a delirium assessment system component 30, and a sepsis assessment system component 22. These components may be implemented as previously described with respect to FIGS. 1 through 5. The pain assessment system component 10, delirium assessment system component 30, and sepsis assessment system component 22 may include devices that receive the scores (or levels) generated by the corresponding systems. According to various implementations, the medication delivery component 14 may provide infusion information including a currently administered analgesic or other medication details, including but not limited to drug and concentration, infusion pump settings currently being utilized to administer medications, and medication (e.g. sedation)sedation levels currently being administered.

The ventilation medication choice and dosing system 60 is configured to receive and score or classify data from each component, and is configured to (based on a predetermined algorithm or neural network) output ventilation medication and dosing recommendations. Upon generating ventilation medication and dosing recommendations, the ventilation medication choice and dosing system may deliver this information as a notification to a caregiver, through either a screen on the mechanical ventilator, via a mobile application on the caregiver's device, via a web application to a network connected device, or other means of notification. In addition to relaying this information to an end-user, in some implementations, a score or value based on a classification of the recommendation may be used as an integral input fed back into patient devices (e.g. ventilator, infusion pump, etc.) to adjust key clinical parameters in an automated fashion, as described further with regard to the example implementation(s) of FIG. 7. When used as input to adjust a patient care device, the assessment system may confirm the adjustment and deliver confirmatory messages or check-in notifications to a caregiver through mobile, web, machine or audible means.

FIG. 7A depicts an example implementation of an automatic management system, according to various aspects of the subject technology. In the depicted example, an automatic risk factor management system 70 receives one or more risk factors and/or scores attributed to various detrimental symptoms of prolonged ICU stays. These risk factors and/or scores are based on data received from the foregoing components described with respect to FIGS. 1 through 4. In this regard, data is received from one or more of a pain assessment system component, a sepsis assessment system component, a delirium assessment system component, and an ICU-acquired weakness assessment system component. The output of each system component may include a discrete score or probability. As described previously, each system component may include its own respective inputs and algorithms used to calculate the corresponding scores/probabilities, which are input to the automatic management system. According to various implementations, the medication delivery component 14 and mechanical ventilator component 18 are utilized as inputs for all or some of the input systems and, thereby may be implicit inputs to the entire automatic risk factor management system 70.

According to various implementations, the automatic risk factor management system 70 is configured to be executed with a pre-determined frequency, and at the beginning of each initiation the current state of the input system components previously described. During execution, the inputs are collectively fed to a Q-learning or reinforcement learning algorithm, which automatically finds the optimal action-based policy to reach a desired goal/outcome. In some implementations, the selected action-based policy is a policy to automatically adjust parameters that influence the operation of a mechanical ventilator or a medication delivery device, for example, via the mechanical ventilator component 18 and/or medication delivery component 14. A policy and its corresponding parameters is selected to keep pain scores, sepsis probabilities, ICUAW scores, and delirium probabilities at a given target value or below a pre-determined threshold. The current state of the patient is updated by an algorithm, and the patient state and/or the updated state of the medical device adjusted by the system is fed back as input(s) to create a closed-loop system.

FIG. 7B depicts an example implementation of a reinforcement learning algorithm for use by the automatic risk factor management system 70, according to various aspects of the subject technology. The algorithm is represented with two parties—an agent and the environment. The environment may include the updated state or condition of one or more devices or the patient (e.g., a physiological state represented by give measurements). In the depicted example, the agent acts on the environment and receives feedback from the environment in terms of a reward for its action and the information of the new state. This reward informs the agent as to how good or poor the action/decision was and determines what the next state in the environment will be. The agent ultimately determines the best series of actions to take in the environment in order to carry out the task at hand in the best possible manner, which in this case is to keep the aforementioned specific risk factors under control (e.g. pain, sepsis, delirium, ICU-acquired weakness). As shown in FIG. 7B, the current states comprise the current scores and probabilities from the system components and the actions comprise specific changes to device setting parameters. In some implementations, the risk factor management system architecture and reinforcement learning algorithm are utilized to both predict and execute weaning of the patient from the ventilator, which would otherwise require a user to initiate and execute. As a result of determining that the patient is a candidate for weaning (including, e.g., extubation), the system may automatically adjust the patient care device with an updated set of parameters to initiate the weaning (e.g., reduce or adjust an amount of ventilation). Updated parameters set by the system may include an infusion pump concentration or dosage, or a reduction in PEEP.

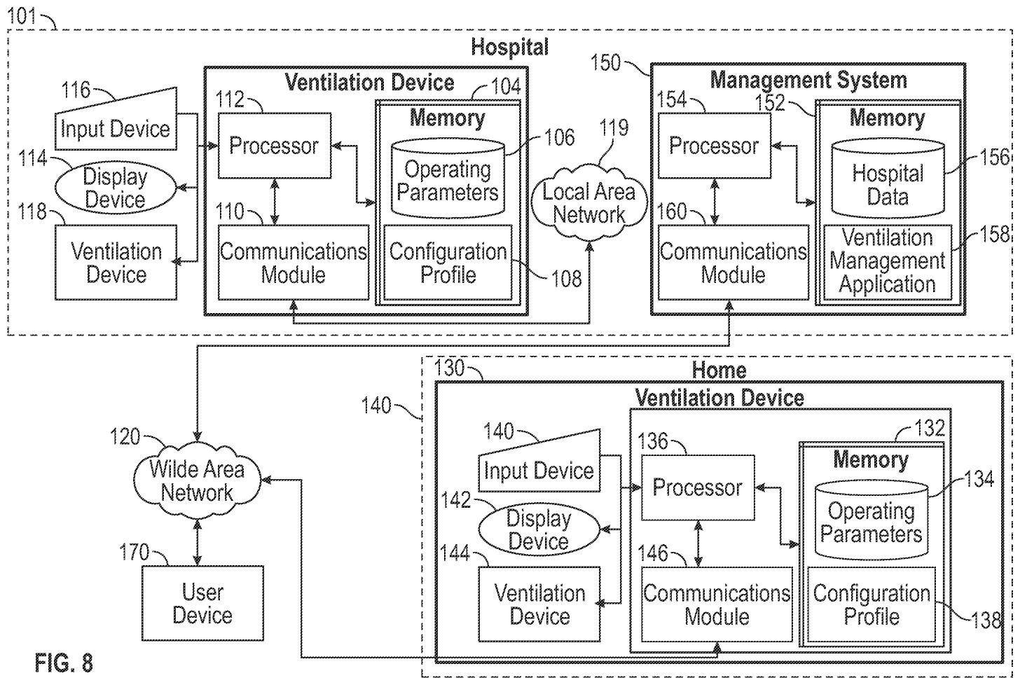

FIG. 8 is a block diagram illustrating an example system for assessing conditions of ventilated patients and adjusting an operation mode of a ventilator, including a ventilation device 102, a management system 150, and a ventilation device 130, according to certain aspects of the subject technology. Management system 150 may include a server and, in many aspects, includes logic and instructions for providing the functionality previously described with regard to FIGS. 1 through 15. For example, a server of management system 150 may broker communications between the various devices, and/or generate user interface 10 for display by user devices 170. Ventilation device 102 and ventilation device 130 may represent each of multiple ventilation devices connected to management system 150. Although the management system 150 is illustrated as connected to a ventilation device 102 and a ventilation device 130, the management system 150 is configured to also connect to different medical devices, including infusion pumps, point of care vital signs monitors, and pulmonary diagnostics devices. In this regard, device 102 or device 130 may be representative of a different medical device.

Ventilation device 102 is connected to the management system 150 over the LAN 119 via respective communications modules 110 and 160 of the ventilation system 102 and the management system 150. The management system 150 is connected over WAN 120 to the ventilation device 130 via respective communications modules 160 and 146 of the management system 150 and the ventilation device 130. The ventilation device 130 is configured to operate substantially similar to the ventilation device 102 of a hospital system 101, except that the ventilation device (or medical device) 130 is configured for use in the home 140. The communications modules 110, 160, and 146 are configured to interface with the networks to send and receive information, such as data, requests, responses, and commands to other devices on the networks. The communications modules 110, 160, and 146 can be, for example, modems, Ethernet cards, or WiFi component modules and devices.

The management system 150 includes a processor 154, the communications module 160, and a memory 152 that includes hospital data 156 and a management application 158. Although one ventilation device 102 is shown in FIG. 16, the management system 150 is configured to connect with and manage many ventilation devices 102, both ventilation devices 102 for hospitals and corresponding systems 101 and ventilation devices 130 for use in the home 140.

In certain aspects, the management system 150 is configured to manage many ventilation devices 102 in the hospital system 101 according to certain rules and procedures. For example, when powering on, a ventilation system 102 may send a handshake message to the management system 150 to establish a connection with the management system 150. Similarly, when powering down, the ventilation system 102 may send a power down message to the management system 150 so that the management system 150 ceases communication attempts with the ventilation system 102.

The management system 150 is configured to support a plurality of simultaneous connections to different ventilation devices 102 and ventilation devices 130, and to manage message distribution among the different devices, including to and from a user device 170. User device 170 may be a mobile device such as a laptop computer, tablet computer, or mobile phone. User device 170 may also be a desktop or terminal device authorized for use by a user. In this regard, user device 170 is configured with the previously described messaging application depicted by FIGS. 1 through 15 to receive messages, notifications, and other information from management system 150, as described throughout this disclosure.

The number of simultaneous connections can be configured by an administrator in order to accommodate network communication limitations (e.g., limited bandwidth availability). After the ventilation device 102 successfully handshakes with (e.g., connects to) the management system 150, the management system 150 may initiate communications to the ventilation device 102 when information becomes available, or at established intervals. The established intervals can be configured by a user so as to ensure that the ventilation device 102 does not exceed an established interval for communicating with the management system 150.

The management system 150 can receive or provide data to the ventilation device 102, for example, to adjust patient care parameters of the ventilation device. For instance, alerts may be received from ventilation device 102 (or device 130) responsive to thresholds being exceeded. An admit-discharge-transfer communication can be sent to specified ventilation devices 102 within a certain care area of a hospital 101. Orders specific to a patient may be sent to a ventilation device 102 associated with the patient, and data specific to a patient may be received from ventilation device 102.

The ventilation device 102 may initiate a communication to the management system 150 if an alarm occurs on the ventilation system 102. The alarm may be indicated as time-sensitive and sent to the beginning of the queue for communicating data to the management system 150. All other data of the ventilation device 102 may be sent together at once, or a subset of the data can be sent at certain intervals.

Hospital data 156 may continuously or periodically received (in real time or near real time) by management system 150 from each ventilator device 102 and each ventilator device 130. The hospital data 156 may include configuration profiles configured to designate operating parameters for a respective ventilation device 102, operating parameters of each ventilation device 102 and/or physiological statistics of a patient associated with the ventilation device 102. Hospital data 156 also includes patient data for patients admitted to a hospital or within a corresponding hospital system 101, order (e.g., medication orders, respiratory therapy orders) data for patients registered with the hospital 101 system, and/or user data (e.g., for caregivers associated with the hospital system 101). As described previously with regard to the systems described with regard to FIGS. 1 through 7, hospital data 156 may be updated or changed based on an updated state provided by these systems.

The physiological statistics and/or measurements of the ventilator data includes, for example, a statistic(s) or measurement(s) indicating compliance of the lung (Cdyn, Cstat), flow resistance of the patient airways (Raw), inverse ratio ventilation (I/E), spontaneous ventilation rate, exhaled tidal volume (Vte), total lung ventilation per minute (Ve), peak expiratory flow rate (PEFR), peak inspiratory flow rate (PIFR), mean airway pressure, peak airway pressure, an average end-tidal expired CO2 and total ventilation rate. The operating parameters include, for example, a ventilation mode, a set mandatory tidal volume, positive end respiratory pressure (PEEP), an apnea interval, a bias flow, a breathing circuit compressible volume, a patient airway type (for example endotracheal tube, tracheostomy tube, face mask) and size, a fraction of inspired oxygen (FiO2), a breath cycle threshold, and a breath trigger threshold.

The processor 154 of the management system 150 is configured to execute instructions, such as instructions physically coded into the processor 154, instructions received from software (e.g., management application 158) in memory 152, or a combination of both. For example, the processor 154 of the management system 150 executes instructions to receive ventilator data from the ventilation device(s) 102 (e.g., including an initial configuration profile for the ventilation system 102).

Ventilation device 102 is configured to send ventilator information, notifications (or “alarms”), scalars, operating parameters 106 (or “settings”), physiological statistics (or “monitors”) of a patient associated with the ventilation device 102, and general information. The notifications include operational conditions of the ventilation device 102 that may require operator review and corrective action. Scalars include parameters that are typically updated periodically (e.g., every 500 ms) and can be represented graphically on a two-dimensional scale. The physiological statistics represent information that the ventilation device 102 is monitoring, and can dynamic based on a specific parameter. The operating parameters 106 represent the operational control values that the caregiver has accepted for the ventilation device 102. The general information can be information that is unique to the ventilation device 102, or that may relate to the patient (e.g., a patient identifier). The general information can include an identifier of the version and model of the ventilation device 102. It is also understood that the same or similar data may be communicated between management system 150 and ventilation device 130.

FIG. 8 further illustrates an example distributed server-client system for providing the disclosed user interface (represented by display screens of FIGS. 1 through 15). Management system 150 may include (among other equipment) a centralized server and at least one data source (e.g., a database 152). The centralized server and data source(s) may include multiple computing devices distributed over a local 119 or wide area network 120, or may be combined in a single device. Data may be stored in data source(s) 152 (e.g., a database) in real time and managed by the centralized server. In this regard, multiple medical devices 102, 130 may communicate patient data, over network 119, 120, to the centralized server in real time as the data is collected or measured from the patient, and the centralized server may store the patient data in data source(s) 152. According to some implementations, one or more servers may receive and store the patient data in multiple data sources.

According to various implementations, management system 150 (including centralized server) is configured to (by way of instructions) generate and provide virtual user interface 10 to clinician devices 170. In some implementations, management system 150 may function as a web server, and virtual interface 100 may rendered from a website provided by management system 150. According to various implementations, management system 150 may aggregate real time patient data and provide the data for display in virtual interface 100. The data and/or virtual interface 100 may be provided (e.g., transmitted) to each clinician device 170, and each clinician device 170 may include a software client program or other instructions configured to, when executed by one or more processors of the device, render and display virtual interface 100 with the corresponding data. The depicted clinician devices 170 may include personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity. While not shown in FIG. 16, it is understood that the connections between the various devices over local network 119 or wide area network 120 may be made via a wireless connection such as WiFi, BLUETOOTH, Radio Frequency, cellular, or other similar connection.

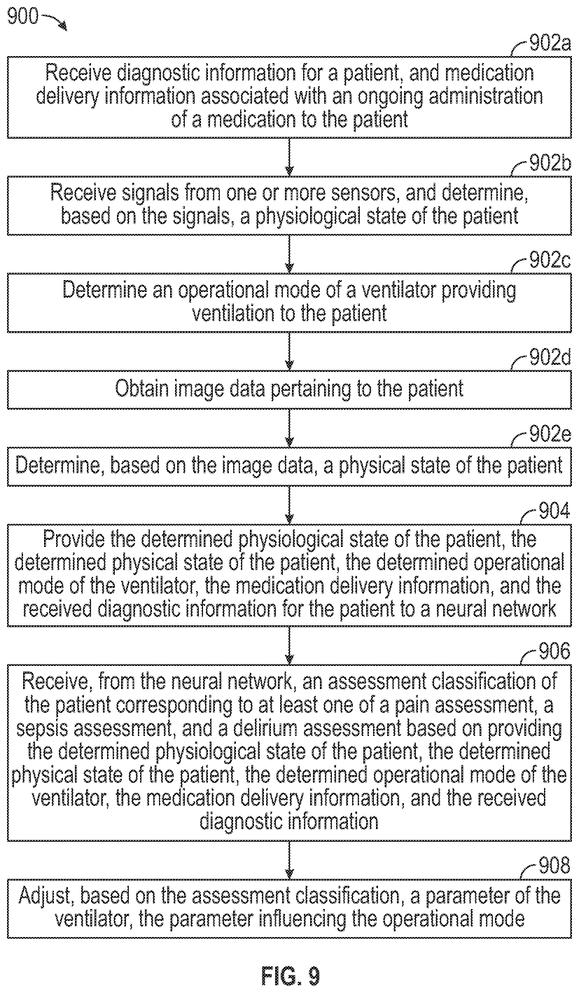

FIG. 9 depicts an example flow chart of a process 900 of assessing conditions of ventilated patients and adjusting an operation mode of a ventilator, according to aspects of the subject technology. The process 900 is implemented, in part, through the exchange of data between the ventilation device 102, the management system 150, and user device 170. For explanatory purposes, the various blocks of example process 900 are described herein with reference to FIGS. 1 through 8, and the components and/or processes described herein. The one or more of the blocks of process 900 may be implemented, for example, by a computing device, including a processor and other components utilized by the device. In some implementations, one or more of the blocks may be implemented apart from other blocks, and by one or more different processors or devices. Further for explanatory purposes, the blocks of example process 900 are described as occurring in serial, or linearly. However, multiple blocks of example process 900 may occur in parallel. In addition, the blocks of example process 900 need not be performed in the order shown and/or one or more of the blocks of example process 900 need not be performed.

The example process may be implemented by a system comprising a ventilation communication device (e.g., device 18) configured to receive ventilation data, a medication delivery communication device (e.g., device 14) configured to receive medication delivery information associated with an ongoing administration of a medication to the patient, an image capture device (e.g., device 12), and one or more sensors configured to obtain physiological data from a patient. The disclosed system may include a memory 152 storing instructions and data 156, and one or more processors 154 configured to execute the instructions to perform operations.

In the depicted example flow diagram, certain information is obtained from the various component devices (902a-e). Diagnostic information is received for the patient by the management system 150, and the management system 150 determines, based on signals received from the one or more sensors, a physiological state of the patient. System 150 determines, from the ventilation communication device, an operational mode of the ventilator. System 150 receives the medication delivery information from the medication delivery communication device. System 150 activates the imaging device and obtain image data pertaining to the patient from the imaging device, and determines, based on the image data, a physical state of the patient. System 150 then provides the determined physiological state of the patient, the determined physical state of the patient, the determined operational mode of the ventilator, the medication delivery information, and the received diagnostic information for the patient to a neural network (904), and receives, from the neural network, an assessment classification of the patient corresponding to at least one of a pain assessment, a sepsis assessment, and a delirium assessment of the patient based on providing to the neural network the determined physiological state of the patient, the determined physical state of the patient, the determined operational mode of the ventilator, the medication delivery information, and the received diagnostic information for the patient (906). The system 150 then adjusts, based on the assessment classification, a parameter of the ventilator 102, 130, wherein adjusting the parameter influences the operational mode of the ventilator (908).

According to various aspects, the image capture device (e.g., a vision component 12) comprises a camera, and the one or more sensors comprises an accelerometer affixed to the patient. In such implementations, management system 150 receives one or more image frames from the camera, and accelerometer data from the accelerometer, and provides the image frames and accelerometer data to a recognition algorithm configured to determine a shivering state or a restlessness state of the patient. Management system 150 determines, by the recognition algorithm, a patient body state indicative of the shivering state or the restlessness state of the patient. With regard to example process 900, the physical state of the patient may include the determined patient body state. In some implementations, the recognition algorithm is configured to determine the shivering state of the patient, wherein the shivering state of the patient is indicated by a numerical value within a range of values representative of the patient being still or calm state to being in an exaggerated shivering state.

Additionally or in the alternative, in some implementations, the image capture device includes a camera configured adjacent to the patient and positioned to capture an image of the patient's face. In this regard, management system 150 may be configured to receive one or more image frames from the camera, and provide the one or more image frames to a facial recognition algorithm configured to recognize features of the patient's face in the one or more images. The algorithm maps the recognized features to a facial state indicative of the patient's facial expression, the determined facial state being representative of one of a relaxed state, a tense state, and a grimacing state. With regard to example process 900, the physical state of the patient may include the determined facial state.

According to various implementations, the one or more sensors may include a sensor applied to the patient's skin and configured to measure a level of muscle tension, wherein the physical state of the patient comprises the level of muscle tension. Additionally or in the alternative, the one or more sensors may include a sensor configured to obtain a vital sign measurement of the patient, including one or more of blood pressure, patient core temperature, heart rate, electrocardiogram (ECG) signal, pulse, or blood oxygen saturation level, wherein the determined physiological state of the patient comprises information representative of the vital sign measurement. In some implementations, the medication delivery communication device (e.g., component 14) is configured to receive, from an infusion pump, the medication delivery information, the medication delivery information comprising a drug identification, drug concentration, drug dosage, or length of an ongoing infusion.

In some implementations, management system 150 (or hospital system 101) is configured to receive diagnostic information for the patient. The diagnostic information may include lab results associated with the patient received from a diagnostic information system. According to various implementations, the system 150 or system 101 further comprises an audio device configured adjacent to the patient and positioned to capture audio from the patient. In this regard, the system may receive patient audio information from the audio device, and provide patient audio information to an audio recognition algorithm configured to recognize an audio pattern within the patient audio information, and configured to map the recognized audio pattern to an audio state indicative of a physical or mental state of the patient. With regard to example process 900, the assessment classification may be further based on the audio state provided to the neural network.

System 150 or system 101 may also include a strength assessment device configured to assess a muscle strength of the patient based on a pressure exerted by the patient on the strength assessment device. In this regard, system 150 may be configured to receive strength information from the strength assessment device, and provide the strength information to a strength assessment algorithm configured to map the strength information to a strength classification indicative of a physical strength of the patient. The strength classification may be provided (e.g., by system 150) to the neural network, and the assessment classification is further based on the strength classification provided to the neural network.

As described previously, the assessment classification may include a pain level of the patient, a sepsis level indicative of a sepsis condition in the patient, a probability that the patient having an intensive care unit-acquired weakness or is suffering from post-intensive care unit syndrome, or a delirium level indicative of a level of delirium of the patient, depending on which data is collected and/or from which components the data is collected. As described previously, the management system 150 may send a message pertaining to the assessment classification and the adjusted parameter to a user device 170, remote from the system 101, 150, for display by a user interface operating on the user device when the user is authenticated to the system via the user interface.

Many aspects of the above-described example 900, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 10 is a conceptual diagram illustrating an example electronic system 1000 for assessing conditions of ventilated patients and adjusting an operation mode of a ventilator, according to aspects of the subject technology. Electronic system 1000 may be a computing device for execution of software associated with one or more portions or steps of process 1000, or components and processes provided by FIGS. 1 through 9. Electronic system 1000 may be representative, in combination with the disclosure regarding FIGS. 1 through 9, of the management system 150 (or server of system 150) or the clinician device(s) 170 described above. In this regard, electronic system 1000 or computing device may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 1000 may include various types of computer readable media and interfaces for various other types of computer readable media. In the depicted example, electronic system 1700 includes a bus 1008, processing unit(s) 1012, a system memory 1004, a read-only memory (ROM) 1010, a permanent storage device 1002, an input device interface 1014, an output device interface 1006, and one or more network interfaces 1016. In some implementations, electronic system 1000 may include or be integrated with other computing devices or circuitry for operation of the various components and processes previously described.

Bus 1008 collectively represents all system, peripheral, and chipset buses that communicatively connect the numerous internal devices of electronic system 1000. For instance, bus 1008 communicatively connects processing unit(s) 1012 with ROM 1010, system memory 1004, and permanent storage device 1002.

From these various memory units, processing unit(s) 1012 retrieves instructions to execute and data to process in order to execute the processes of the subject disclosure. The processing unit(s) can be a single processor or a multi-core processor in different implementations.

ROM 1010 stores static data and instructions that are needed by processing unit(s) 1012 and other modules of the electronic system. Permanent storage device 1002, on the other hand, is a read-and-write memory device. This device is a non-volatile memory unit that stores instructions and data even when electronic system 1000 is off. Some implementations of the subject disclosure use a mass-storage device (such as a magnetic or optical disk and its corresponding disk drive) as permanent storage device 1002.

Other implementations use a removable storage device (such as a floppy disk, flash drive, and its corresponding disk drive) as permanent storage device 1002. Like permanent storage device 1002, system memory 1004 is a read-and-write memory device. However, unlike storage device 1002, system memory 1004 is a volatile read-and-write memory, such a random access memory. System memory 1004 stores some of the instructions and data that the processor needs at runtime. In some implementations, the processes of the subject disclosure are stored in system memory 1004, permanent storage device 1002, and/or ROM 1010. From these various memory units, processing unit(s) 1012 retrieves instructions to execute and data to process in order to execute the processes of some implementations.

Bus 1008 also connects to input and output device interfaces 1014 and 1006. Input device interface 1014 enables the user to communicate information and select commands to the electronic system. Input devices used with input device interface 1014 include, e.g., alphanumeric keyboards and pointing devices (also called “cursor control devices”). Output device interfaces 1006 enables, e.g., the display of images generated by the electronic system 1000. Output devices used with output device interface 1006 include, e.g., printers and display devices, such as cathode ray tubes (CRT) or liquid crystal displays (LCD). Some implementations include devices such as a touchscreen that functions as both input and output devices.

Also, as shown in FIG. 10, bus 1008 also couples electronic system 1700 to a network (not shown) through network interfaces 1016. Network interfaces 1016 may include, e.g., a wireless access point (e.g., Bluetooth or WiFi) or radio circuitry for connecting to a wireless access point. Network interfaces 1016 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 1700 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer,” “server,” “processor,” and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A system, comprising: a ventilation communication device configured to receive ventilation data; a medication delivery communication device configured to receive medication delivery information associated with an ongoing administration of a medication to the patient; an image capture device; one or more sensors; a memory storing instructions; and one or more processors configured to execute the instructions to perform operations comprising: receiving diagnostic information for the patient; determining, based on signals received from the one or more sensors, a physiological state of the patient; determining, from the ventilation communication device, an operational mode of the ventilator; receiving the medication delivery information from the medication delivery communication device; activating the imaging device and obtain image data pertaining to the patient from the imaging device; determining, based on the image data, a physical state of the patient; providing the determined physiological state of the patient, the determined physical state of the patient, the determined operational mode of the ventilator, the medication delivery information, and the received diagnostic information for the patient to a neural network; receiving, from the neural network, an assessment classification of the patient corresponding to at least one of a pain assessment, a sepsis assessment, and a delirium assessment of the patient based on providing to the neural network the determined physiological state of the patient, the determined physical state of the patient, the determined operational mode of the ventilator, the medication delivery information, and the received diagnostic information for the patient; and adjusting, based on the assessment classification, a parameter of the ventilator, wherein adjusting the parameter influences the operational mode of the ventilator.

Clause 2. The system of Clause 1, wherein the image capture device comprises a camera, and the one or more sensors comprises an accelerometer affixed to the patient, wherein the operations further comprise: receiving one or more image frames from the camera, and accelerometer data from the accelerometer, providing the image frames and accelerometer data to a recognition algorithm configured to determine a shivering state or a restlessness state of the patient; and determining, by the recognition algorithm, a patient body state indicative of the shivering state or the restlessness state of the patient, wherein the physical state of the patient comprises the determined patient body state.

Clause 3. The system of Clause 2, wherein the recognition algorithm is configured to determine the shivering state of the patient, wherein the shivering state of the patient is indicated by a numerical value within a range of values representative of the patient being still or calm state to being in an exaggerated shivering state.

Clause 4. The system of any of the preceding Clauses, wherein the image capture device comprises a camera configured adjacent to the patient and positioned to capture an image of the patient's face, wherein the operations further comprise: receiving one or more image frames from the camera; providing the one or more image frames to a facial recognition algorithm configured to recognize features of the patient's face in the one or more images, and to map the recognized features to a facial state indicative of the patient's facial expression, the determined facial state being representative of one of a relaxed state, a tense state, and a grimacing state, wherein the physical state of the patient comprises the determined facial state.

Clause 5. The system of any of the preceding Clauses, wherein the one or more sensors comprises a sensor applied to the patient's skin and configured to measure a level of muscle tension, wherein the physical state of the patient comprises the level of muscle tension.

Clause 6. The system of any of the preceding Clauses, wherein the one or more sensors comprises a sensor configured to obtain a vital sign measurement of the patient, including one or more of blood pressure, patient core temperature, heart rate, electrocardiogram (ECG) signal, pulse, or blood oxygen saturation level, wherein the determined physiological state of the patient comprises information representative of the vital sign measurement.

Clause 7. The system of any of the preceding Clauses, wherein the medication delivery communication device is configured to receive, from an infusion pump, the medication delivery information, the medication delivery information comprising a drug identification, drug concentration, drug dosage, or length of an ongoing infusion.

Clause 8. The system of any of the preceding Clauses, wherein the assessment classification comprises a pain level of the patient.

Clause 9. The system of any of the preceding Clauses, wherein receiving diagnostic information for the patient comprises receiving lab results associated with the patient from an diagnostic information system.

Clause 10. The system of any of the preceding Clauses, wherein the assessment classification comprises a sepsis level indicative of a sepsis condition in the patient.

Clause 11. The system of any of the preceding Clauses, wherein the system further comprises an audio device configured adjacent to the patient and positioned to capture audio from the patient, wherein the operations further comprise: receiving patient audio information from the audio device; and providing patient audio information to an audio recognition algorithm configured to recognize an audio pattern within the patient audio information, and to map the recognized audio pattern to an audio state indicative of a physical or mental state of the patient, wherein the audio state is provided to the neural network, and the assessment classification is further based on the audio state provided to the neural network.

Clause 12. The system of any of the preceding Clauses, wherein the system further comprises a strength assessment device configured to assess a muscle strength of the patient based on a pressure exerted by the patient on the strength assessment device, wherein the operations further comprise: receiving strength information from the strength assessment device; and providing the strength information to a strength assessment algorithm configured to map the strength information to a strength classification indicative of a physical strength of the patient, wherein the strength classification is provided to the neural network, and the assessment classification is further based on the strength classification provided to the neural network.

Clause 13. The system of any of the preceding Clauses, wherein the assessment classification comprises a probability that the patient having an intensive care unit-acquired weakness or is suffering from post-intensive care unit syndrome.

Clause 14. The system of any of the preceding Clauses, wherein the assessment classification comprises a delirium level indicative of a level of delirium of the patient.

Clause 15. The system of any of the preceding Clauses, wherein the operations further comprise: sending a message pertaining to the assessment classification and the adjusted parameter to a user device, remote from the system, for display by a user interface operating on the user device when the user is authenticated to the system via the user interface.

Clause 16. A non-transitory computer-readable medium comprising instructions, which when executed by a computing device, cause the computing device to perform operations comprising: receiving diagnostic information for a patient; determining, based on signals received from one or more sensors, a physiological state of the patient; determining an operational mode of a ventilator providing ventilation to the patient; receiving medication delivery information from medication delivery device; activating an imaging device and obtaining image data pertaining to the patient from the imaging device; determining, based on the image data, a physical state of the patient; providing the determined physiological state of the patient, the determined physical state of the patient, the determined operational mode of the ventilator, the medication delivery information, and the received diagnostic information for the patient to a neural network; receiving, from the neural network, an assessment classification of the patient corresponding to at least one of a pain assessment, a sepsis assessment, and a delirium assessment of the patient based on providing to the neural network the determined physiological state of the patient, the determined physical state of the patient, the determined operational mode of the ventilator, the medication delivery information, and the received diagnostic information for the patient; and adjusting, based on the assessment classification, a parameter of the ventilator, wherein adjusting the parameter influences the operational mode of the ventilator.

Clause 17. The non-transitory computer-readable medium of Clause 16, wherein the image capture device comprises a camera, and the one or more sensors comprises an accelerometer affixed to the patient, wherein the operations further comprise: receiving one or more image frames from the camera, and accelerometer data from the accelerometer, providing the image frames and accelerometer data to a recognition algorithm configured to determine a shivering state or a restlessness state of the patient; and determining, by the recognition algorithm, a patient body state indicative of the shivering state or the restlessness state of the patient, wherein the physical state of the patient comprises the determined patient body state.

Clause 18. The non-transitory computer-readable medium of any of the preceding Clauses, wherein the image capture device comprises a camera configured adjacent to the patient and positioned to capture an image of the patient's face, wherein the operations further comprise: receiving one or more image frames from the camera; providing the one or more image frames to a facial recognition algorithm configured to recognize features of the patient's face in the one or more images, and to map the recognized features to a facial state indicative of the patient's facial expression, the determined facial state being representative of one of a relaxed state, a tense state, and a grimacing state, wherein the physical state of the patient comprises the determined facial state.

Clause 19. A method for assessing a condition of a ventilated patient and adjusting an operation mode of the ventilator, comprising: receiving diagnostic information for a patient; receiving, from a medication delivery device, medication delivery information associated with an ongoing administration of a medication to the patient; determining, based on signals received from one or more sensors, a physiological state of the patient; determining an operational mode of a ventilator providing ventilation to the patient; activating an imaging device and obtaining image data pertaining to the patient from the imaging device; determining, based on the image data, a physical state of the patient; providing the determined physiological state of the patient, the determined physical state of the patient, the determined operational mode of the ventilator, the medication delivery information, and the received diagnostic information for the patient to a neural network; receiving, from the neural network, an assessment classification of the patient corresponding to at least one of a pain assessment, a sepsis assessment, and a delirium assessment of the patient based on providing to the neural network the determined physiological state of the patient, the determined physical state of the patient, the determined operational mode of the ventilator, the medication delivery information, and the received diagnostic information for the patient; and adjusting, based on the assessment classification, a parameter of the ventilator, wherein adjusting the parameter influences the operational mode of the ventilator.

Clause 20. The method of Clause 19, wherein the image capture device comprises a camera, and the one or more sensors comprises an accelerometer affixed to the patient, the method further comprising: receiving one or more image frames from the camera, and accelerometer data from the accelerometer, providing the image frames and accelerometer data to a recognition algorithm configured to determine a shivering state or a restlessness state of the patient; and determining, by the recognition algorithm, a patient body state indicative of the shivering state or the restlessness state of the patient, wherein the physical state of the patient comprises the determined patient body state.

Further Consideration:

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit this disclosure.

The term website, as used herein, may include any aspect of a website, including one or more web pages, one or more servers used to host or store web related content, etc. Accordingly, the term website may be used interchangeably with the terms web page and server. The predicate words “configured to,” “operable to,” and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U. S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

1-20. (canceled)

21. A system, comprising:
an image capture device configured to capture an image of a patient associated with a ventilator;
one or more processors configured to:
receive sensor data from one or more sensors associated with the patient;
determine, based on the sensor data, a physiological state of the patient;
receive, from the image capture device, image data associated with the patient;
determine, based on the image data, a physical state of the patient;
identify an operational mode of the ventilator;
determine an assessment classification indicating whether the patient is in a state of pain, sepsis, or a delirium based on the determined physiological state of the patient, the determined physical state of the patient, and the operational mode of the ventilator; and
adjust, based on the assessment classification, one or more operating parameters of the ventilator, wherein adjusting the one or more operating parameters influences the operational mode of the ventilator.

22. The system of claim 21, the one or more processors being further configured to:
receiving diagnostic information associated with the patient; and
determine the assessment classification based on the diagnostic information, determined physiological state of the patient, the determined physical state of the patient, and the operational mode of the ventilator.

23. The system of claim 21, the one or more processors being further configured to:
receive medication delivery information associated with a medication being administered to the patient; and
determine the assessment classification based on the medication delivery information, determined physiological state of the patient, the determined physical state of the patient, and the operational mode of the ventilator.

24. The system of claim 21, the one or more processors being further configured to:
receive accelerometer data associated with at least one accelerometer coupled to the patient;
detect one or more motions of the patient based on the accelerometer data;
determine a classification of the one or more motions; and
determine the physical state of the patient based on the classification of the one or more motions and the image data.

25. The system of claim 21, wherein the operating parameters include, for example, a ventilation mode, a set mandatory tidal volume, positive end respiratory pressure (PEEP), an apnea interval, a bias flow, a breathing circuit compressible volume, a patient airway type or size, a fraction of inspired oxygen (Fi02), a breath cycle threshold, or a breath trigger threshold.

26. The system of claim 21, the one or more processors being further configured to:
adjust the one or more operating parameters to initiate a ventilation weaning,
wherein the one or more operating parameters include a concentration or dosage of a medication administered to the patient by an infusion pump, or a reduction in positive end respiratory pressure (PEEP) provided by the ventilator.

27. The system of claim 21, wherein the image capture device is a camera configured adjacent to the patient and positioned to capture at least one image of the patient's face, wherein the one or more processors are further configured to:
receive the one or more images from the camera; and
provide the one or more images to a facial recognition algorithm configured to recognize facial features and map the facial features to a facial state comprising one of a relaxed state, a tense state, and a grimacing state,
wherein the physical state of the patient is further determined based on the determined facial state.

28. The system of claim 21, wherein the one or more sensors comprises a sensor applied to the patient's skin and configured to measure a level of muscle tension, wherein the physical state of the patient is further determined based on the level of muscle tension.

29. The system of claim 21, wherein the one or more sensors comprises a sensor configured to obtain a vital sign measurement of the patient, including one or more of blood pressure, patient core temperature, heart rate, electrocardiogram (ECG) signal, pulse, or blood oxygen saturation level, wherein the determined physiological state of the patient comprises information representative of the vital sign measurement.

30. The system of claim 21, wherein the system further comprises:
an audio device positioned to capture audio from the patient, the one or more processors being further configured to:
receive audio information associated with the patient from the audio device; and
provide the audio information to an audio recognition algorithm configured to recognize an audio pattern and map the recognized audio pattern to an audio state indicative of a physical or mental state of the patient,
wherein the assessment classification is further based on the audio state.

31. The system of claim 21, wherein the system further comprises a strength assessment device configured to assess a muscle strength of the patient based on a pressure exerted by the patient on the strength assessment device, the one or more processors being further configured to:
receive strength information associated with the patient from the strength assessment device; and
map the strength information to a strength classification indicative of a physical strength of the patient,
wherein the assessment classification is further based on the strength classification.

32. The system of claim 21, the one or more processors being further configured to:
send a message pertaining to the assessment classification and the adjusted one or more operating parameters to a user device, remote from the system, for display by a user interface operating on the user device when a user associated with the user device is authenticated to the system via the user interface.

33. A machine-implemented method, comprising:
receiving sensor data from one or more sensors associated with a patient provided ventilation by a ventilator;
determining, based on the sensor data, a physiological state of the patient;
receiving, from an image capture device, image data associated with the patient;
determining, based on the image data, a physical state of the patient;
identifying an operational mode of the ventilator;
determining an assessment classification indicating whether the patient is in a state of pain, sepsis, or a delirium based on the determined physiological state of the patient, the determined physical state of the patient, and the operational mode of the ventilator; and
adjusting, based on the assessment classification, one or more operating parameters of the ventilator, wherein adjusting the one or more operating parameters influences the operational mode of the ventilator.

34. The machine-implemented method of claim 33, further comprising:
receiving diagnostic information associated with the patient; and
determining the assessment classification based on the diagnostic information, determined physiological state of the patient, the determined physical state of the patient, and the operational mode of the ventilator.

35. The machine-implemented method of claim 33, further comprising:
receiving medication delivery information associated with a medication being administered to the patient; and
determining the assessment classification based on the medication delivery information, determined physiological state of the patient, the determined physical state of the patient, and the operational mode of the ventilator.

36. The machine-implemented method of claim 33, further comprising:
receiving accelerometer data associated with at least one accelerometer coupled to the patient;
detecting one or more motions of the patient based on the accelerometer data;
determining a classification of the one or more motions; and
determining the physical state of the patient based on the classification of the one or more motions and the image data.

37. The machine-implemented method of claim 33, wherein the image capture device is a camera configured adjacent to the patient and positioned to capture at least one image of the patient's face, the method further comprising:
receiving the one or more images from the camera; and
providing the one or more images to a facial recognition algorithm configured to recognize facial features and map the facial features to a facial state comprising one of a relaxed state, a tense state, and a grimacing state,
wherein the physical state of the patient is further determined based on the determined facial state.

38. The machine-implemented method of claim 33, further comprising:
receiving audio information associated with the patient from an audio device positioned to capture audio from the patient; and
providing the audio information to an audio recognition algorithm configured to recognize an audio pattern and map the recognized audio pattern to an audio state indicative of a physical or mental state of the patient,
wherein the assessment classification is further based on the audio state.

39. The machine-implemented method of claim 33, further comprising:
receive strength information associated with the patient from a strength assessment device configured to assess a muscle strength of the patient based on a pressure exerted by the patient on the strength assessment device; and
map the strength information to a strength classification indicative of a physical strength of the patient,
wherein the assessment classification is further based on the strength classification.

40. A non-transitory machine-readable storage medium having instructions thereon that, when executed by a machine, cause the machine to perform a method comprising:
receiving sensor data from one or more sensors associated with a patient provided ventilation by a ventilator;
determining, based on the sensor data, a physiological state of the patient;
receiving, from an image capture device, image data associated with the patient;
determining, based on the image data, a physical state of the patient;
identifying an operational mode of the ventilator;
determining an assessment classification indicating whether the patient is in a state of pain, sepsis, or a delirium based on the determined physiological state of the patient, the determined physical state of the patient, and the operational mode of the ventilator; and
adjusting, based on the assessment classification, one or more operating parameters of the ventilator, wherein adjusting the one or more operating parameters influences the operational mode of the ventilator.

41. A machine-implemented method, comprising:
receiving diagnostic information for a patient;
receiving, from a medication delivery device, medication delivery information associated with an ongoing administration of a medication to the patient;
determining, based on signals received from one or more sensors, a physiological state of the patient;
determining an operational mode of a ventilator providing ventilation to the patient;
activating a camera to obtain image data pertaining to the patient from the camera;
receiving one or more images from the camera;
providing the one or more images frames to a recognition algorithm configured to determine a physical state of the patient, the physical state including one of shivering or restlessness;
determining, by the recognition algorithm, whether the patient is in the physical state of shivering or restlessness;
providing the determined physiological state of the patient, the determined physical state of the patient, the determined operational mode of the ventilator, the medication delivery information, and the received diagnostic information for the patient to a neural network;
receiving, from the neural network, an assessment classification indicating whether the patient is in a state of pain, sepsis, or a delirium based on providing to the neural network the determined physiological state of the patient, the determined physical state of the patient, the determined operational mode of the ventilator, the medication delivery information, and the received diagnostic information for the patient; and
adjusting, based on the assessment classification, one or more operating parameters of the ventilator, wherein adjusting the one or more operating parameters influences the operational mode of the ventilator.
